(19) United States
(12) Patent Application Publication
(10) Pub. No.: US 2010/0004584 A1
(43) Pub. Date: Jan. 7, 2010

(54) Apparatus and Method for In Vivo Intracellular Transfection of Gene, SIRNA, SHRNA Vectors, and Other Biomedical Diagnostic and Therapeutic Drugs and Molecules for the Treatment of Arthritis and Other Orthopedic Diseases in Large Animals and Humans

(75) Inventor: Luyi Sen, Stevenson Ranch, CA (US)

Correspondence Address: Law Offices of Daniel L. Dawes;Dawes Patent Law Group, 5200 Warner Blvd, Ste. 106, Huntington Beach, CA, 92649, US

(73) Assignee: THE REGENTS OF THE UNIVERSITY OF CALIFORNIA, Oakland, CA (US)

(21) Appl. No.: 12/519,509

(22) PCT Filed: Jan. 2, 2008
(86) PCT No.: PCT/US08/50059

§ 371 Date: Jun. 16, 2009

(51) Int. Cl. A61N 1/30

(52) U.S. Cl. 604/20

(57) ABSTRACT

An apparatus for in vivo intracellular transfection of gene, siRNA, shRNA vectors, and other biomedical diagnostic and therapeutic drugs and molecules for the treatment of arthritis and other orthopedic diseases in large animals and humans includes: a source of low voltage, short duration pulses in long duration bursts (LSEN); an electrode mesh system coupled to the source for generating distributed electric field network into a joint, including bones, cartilages, and related tissues; and means for transfecting the gene, siRNA, shRNA vectors, and other biomedical diagnostic and therapeutic drugs and molecules into a joint. The electrode mesh system includes alternatively arranged negative and positive electrodes in a first array which is capable of being inserted into a joint cavity, and either an alternatively arranged negative and positive or an all negative second electrode array which is positioned outside of the joint and in directly contact with overlying skin.

### RELATED APPLICATIONS

The present application is related to U.S. Provisional Patent Application, Ser. No. 60/883,238, filed on Jan. 3, 2007, which is incorporated herein by reference and to which priority is claimed pursuant to 35 USC 119.

### BACKGROUND OF THE INVENTION

1. Field of the Invention

The invention relates to an apparatus and methodology for highly efficient low strength electric field network-mediated in vivo intracellular transfection of gene, siRNA, shRNA vector, and other biomedical diagnostic and therapeutic drugs and molecules for the treatment of arthritis and other orthopedic diseases in large animals and humans.

2. Description of the Prior Art

Furthermore, more than 80% of drugs act intracellarly, or function by regulating intracellular molecules. Effective gene therapy relies on delivering the nuclear acid into the cell to be effective. siRNA or shRNA are all need to be delivered into cells to be able to function. The efficient intracellular gene, siRNA, and shRNA vector strategy is the major obstacle in their effective clinical application. So far only viral vectors are efficient for gene transfer; however, viral vectors may be toxic and have many side effects that often prevent its clinical use. A clinical applicable safe and efficient in vivo gene delivery method is urgently needed to reopen the door to the promise of gene therapy. To date, still no method is available for in vivo siRNA and shRNA delivery in an animal or human, because these two lines of molecule are even more difficult to deliver in a stable and efficient manner.

Electroporation is a technique involving the application of short duration, high intensity electric field pulses to cells or tissue. The electrical stimulus causes membrane destabilization and the subsequent formation of nanometer-sized pores in the cellular membrane. In this permeabilized state, the membrane can allow passage of DNA, enzymes, antibodies and other macromolecules into the cell. Electroporation holds potential not only in gene therapy, but also in other areas such as transdermal drug delivery and enhanced chemotherapy.

Since the early 1980s, electroporation has been used as a research tool for introducing DNA, RNA, proteins, other macromolecules, liposomes, latex beads, or whole virus particles into living cells. Electroporation efficiently introduces foreign genes into living cells, but the use of this technique had been restricted to suspensions of cultured cells only, since the electric pulse are administered in a cuvette type electrodes.

Electroporation is commonly used for in vitro gene transfection of cell lines and primary cultures, but limited work has been reported in tissue. In one study, electroporation-mediated gene transfer was demonstrated in rat brain tumor tissue. Plasmid DNA was injected intraarterially immediately following electroporation of the tissue. Three days after shock treatment expression of the lacZ gene or the human monocyte chemoattractant protein-1 (MCP-1) gene was detected in electroporated tumor tissue between the two electrodes, but not in adjacent tissue. Electroporation has also been used as a tissue-targeted method of gene delivery in rat liver tissue. This study showed that the transfer of genetic markers β-glactosidase (β-gal) and luciferase resulted in maximal expression at 48 h, with about 30-40% of the electroporated cells expressing β-gal, and luciferase activities reaching peak levels of about 2500 pg/mg of tissue. In another study, electroporation of early chicken embryos was compared to two other transfection methods: microparticle bombardment and lipofection. Of the three transfection techniques, electroporation yielded the strongest intensity of gene expression and extended to the largest area of the embryo. Most recently, an electroporation catheter has been used for delivery heparin to the rabbit arterial wall, and significantly increased the drug delivery efficiency.

Electric pulses with moderate electric field intensity can cause temporary cell membrane permeabilization (cell discharge), which may then lead to rapid genetic transformation and manipulation in wide variety of cell types including bacteria, yeasts, animal and human cells, and so forth. On the other hand, electric pulses with high electric field intensity can cause permanent cell membrane breakdown (cell lysis). According all the knowledge available now, the voltage applied to any tissue must be as high as 100-200 V/cm. If electroporation is to be used on large animal or human organ, such as human heart, it must be supplied at magnitudes of several kV. Such voltage gradients will cause enormous tissue damage. Therefore, this technique is still not applicable for clinical use.

### BRIEF SUMMARY OF THE INVENTION

The illustrated embodiment of the invention is an apparatus for in vivo intracellular transfection of gene, siRNA, shRNA vectors, and other biomedical diagnostic and therapeutic drugs and molecules for the treatment of arthritis and other orthopedic diseases in large animals and humans comprising: a source of low voltage, short duration pulses in long duration bursts (LSEN); an electrode mesh system coupled to the source for generating distributed electric field network into a joint, including bones, cartilages, and related tissues; and means for transfecting the gene, siRNA, shRNA vectors, and other biomedical diagnostic and therapeutic drugs and molecules into a joint. The electrode mesh system comprises alternatively arranged negative and positive electrodes in a first array which is capable of being inserted into a joint cavity, and either an alternatively arranged negative and positive or an all negative second electrode array which is positioned outside of the joint and in directly contact with overlying skin.

In one embodiment the electrode mesh system is capable of being deployed for a chronic treatment period.

In another embodiment the apparatus further comprises an all negative second electrode array positioned on the outside of the joint, and the means for transfecting comprises a slow drug infusion bag or other agent for releasing materials coupled to the first electrode array.

In still another embodiment the apparatus for in vivo intracellular transfection of gene, siRNA, shRNA vectors, and other biomedical diagnostic and therapeutic drugs and molecules for the treatment of arthritis and other orthopedic diseases in large animals and humans comprises: a source of low voltage, short duration pulses in long duration bursts (LSEN); an electrode mesh system coupled to the source for generating distributed electric field network into a spine; and means for transfecting the gene, siRNA, shRNA vectors, and other biomedical diagnostic and therapeutic drugs and molecules into the spine. The electrode mesh system comprises alternatively arranged negative and positive electrodes in a first array which is inserted into a vertebral canal associated with the spine. The means for transfecting comprises a slow drug infusion bag or other agent for releasing materials coupled to the electrode array and also used to shield or insulate the spine from the electric field network.

In another embodiment the illustrated apparatus for in vivo intracellular transfection of gene, siRNA, shRNA vectors, and other biomedical diagnostic and therapeutic drugs and molecules for the treatment of arthritis and other orthopedic diseases in large animals and humans comprises: a source of low voltage, short duration pulses in long duration bursts (LSEN); an electrode mesh system coupled to the source for generating distributed electric field network into a skull or flat bone; and means for transfecting the gene, siRNA, shRNA vectors, and other biomedical diagnostic and therapeutic drugs and molecules into the skull or flat bone. The electrode mesh system comprises either an alternatively arranged negative and positive electrodes in a first array capable of being placed on the skull or flat bone, or an all negative electrode second array is applied on the outside of the skull or flat bone and an all positive first electrode array on the cranial side of the skull or internal side of the flat bone. The means for transfecting comprises a slow drug infusion bag or other agent for releasing materials coupled to the first electrode array and also to shield or insulate the brain from the electric field network in the case of use on the skull.

In yet another embodiment of the invention the apparatus for in vivo intracellular transfection of gene, siRNA, shRNA vectors, and other biomedical diagnostic and therapeutic drugs and molecules for the treatment of arthritis and other orthopedic diseases in large animals and humans comprises: a source of low voltage, short duration pulses in long duration bursts (LSEN); an electrode mesh system coupled to the source for generating distributed electric field network into long bones or joints with screws, needles, prosthesis or other artificial material; and means for transfecting the gene, siRNA, shRNA vectors, and other biomedical diagnostic and therapeutic drugs and molecules into the long bones or joints with screws, needles, prosthesis or other artificial material. The electrode mesh system is comprised of a alternatively arranged negative and positive electrodes in a first array capable of being placed in or on the bones and joints in the position where the screw, needle, prosthesis or other artificial material will be inserted, and an all negative second electrode array positioned on the outside of the joint or long bone. The means for transfecting comprises a slow drug infusion bag or other agent for releasing materials coupled to the first electrode array.

The means for transfecting includes selected molecules effective for the arthritis and other orthopedic diseases and their inhibitors, enhancers, regulators, genes, siRNAs, shRNAs, antigens, antibodies, or peptides related with these molecules.

In particular the selected molecules effective for the arthritis and other orthopedic diseases and their inhibitors, enhancers, regulators, genes, siRNAs, shRNA s, antigens, antibodies, or peptides related with these molecules comprise at least one of:

Cytokines:

i. Chemokines: CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCL3L1, CCL4, CCL4L1, CCL5, CCL7, CCL8, CKLF, CX3CL1, CXCL1, CXCL10, CXCL11, CXCL12, CXCL13, CXCL14, CXCL2, CXCL3, CXCL5, CXCL6, CXCL9, CYP26B1, IL13, IL8, PF4V1, PPBP, PXMP2, XCL1.
ii. Other Cytokines: AREG, BMP1, BMP2, BMP3, BMP7, CAST, CD40LG, CER1, CKLFSF1, CKLFSF2, CLC, CSF1, CSF2, CSF3, CTF1, CXCL16, EBI3, ECGF1, EDA, EPO, ERBB2, ERBB2IP, FAM3B, FASLG, FGF10, FGF12, FIGF, FLT3LG, GDF2, GDF3, GDF5, GDF6, GDF8, GDF9, GLMN, GPI, GREM1, GREM2, GRN, IFNA1, IFNA14, IFNA2, IFNA4, IFNA8, IFNB1, IFNE1, IFNG, IFNK, IFNW1, IFNWP2, IK, IL10, IL11, IL12A, IL12B, IL15, IL16, IL17, IL17B, IL17C, IL17D, IL17E, IL17F, IL18, IL19, IL1A, IL1B, IL1F10, IL1F5, IL1E6, IL1F7, IL1F8, IL1F9, IL1RN, IL2, IL20, IL21, IL22, IL23A, IL24, IL26, IL27, IL28B, IL29, IL3, IL32, IL4, IL5, IL6, IL7, IL9, INHA, INHBA, INHBB, KITLG, LASS1, LEFTY1, LEFTY2, LIF, LTA, LTB, MDK, MIF, MUC4, NODAL, OSM, PBEF1, PDGFA, PDGFB, PRL, PTN, SCGB1A1, SCGB3A1, SCYE1, SDCBP, SECTM1, SIVA, SLCO1A2, SLURP1, SOCS2, SPP1, SPRED1, SRGAP1, THPO, TNF, TNFRSF11B, TNFSF10, TNFSF11, TNFSF13, TNFSF13B, TNFSF14, TNFSF15, TNFSF18, TNFSF4, TNFSF7, TNFSF8, TNFSF9, TRAP1, VEGF, VEGFB, YARS.

Cytokine Receptors:

i. Cytokine Receptors: CNTFR, CSF2RA, CSF2RB, CSF3R, EBI3, EPOR, F3, GFRA1, GFRA2, GHR, IFNAR1, IFNAR2, IFNGR1, IFNGR2, IL10RA, IL10RB, IL11RA, IL12B, IL12RB1, IL12RB2, IL13RA1, IL13RA2, IL15RA, IL17R, IL17RB, IL18R1, IL1R1, IL1R2, IL1RAP, IL1RAPL2, IL1RL1, IL1RL2, IL20RA, IL21R, IL22RA1, IL22RA2, IL28RA, IL2RA, IL2RB, IL2RG, IL31RA, IL3RA, IL4R, IL5RA, IL6R, IL6ST, IL7R, IL8RA, IL8RB, IL9R, LEPR, LIFR, MPL, OSMR, PRLR, TTN.
ii. Chemokine Rectors: BLR1, CCL13, CCR1, CCR10, CCR2, CCR3, CCR4, CCR5, CCR6, CCR7, CCR8, CCR9, CCRL1, CCRL2, CX3CR1, CXCR3, CXCR4, CXCR6, IL8RA, IL8RB, XCR1.

Cytokine Metabolism: APOA2, ASB1, AZU1, B7H3, CD28, CD4, CD80, CD86, EBI3, GLMN, IL10, IL12B, IL17F, IL18, IL21, IL27, IL4, INHA, INHBA, INHBB, IRF4, NALP12, PRG3, S100B, SFTPD, SIGIRR, SPN, TLR1, TLR3, TLR4, TLR6, TNFRSF7, TNFSF15.

Cytokine Production: APOA2, ASB1, AZU1, B7H3, CD28, CD4, CD80, CD86, EBI3, GLMN, IL10, IL12B, IL17F, IL18, IL21, IL27, lL4, INHA, INHBA, INHBB, INS, IRF4, NALP12, NFAM1, NOX5, PRG3, S100B, SAA2, SFTPD, SIGIRR, SPN, TLR1, TLR3, TLR4, TLR6, TNFRSF7.

Other Genes involved in Cytokine-Cytokine Receptor Interaction: ACVR1, ACVR1B, ACVR2, ACVR2B, AMH, AMHR2, BMPR1A, BMPR1B, BMPR2, CCR1, CD40, CRLF2, CSF1R, CXCR3, IL18RAP, IL23R, LEP, TGFB1, TGFB2, TGFB3, TGFBR1, TGFBR2, TNFRSF1A, TNFRSF1B, TNFRSF21, TNFRSF8, TNFRSF9, XCR1.

Acute-Phase Response: AHSG, APCS, APOL2, CEBPB, CRP, F2, F8, FN1, IL22, IL6, INS, ITIH4, LBP, PAP, REG-III, SAA2, SAA3P, SAA4, SERPINA1, SERPINA3, SERPINF2, SIGIRR, STAT3.

Inflammatory Response: ADORA1, AHSG, AIF1, ALOX5, ANXA1, APOA2, APOL3, ATRN, AZU1, BCL6, BDKRB1, BLNK, C3, C3AR1, C4A, CCL1, CCL11, CCL13, CCL16, CCL17, CCL18, CCL19, CCL2, CCL20, CCL21, CCL22, CCL23, CCL24, CCL25, CCL26, CCL3, CCL3L1, CCL4, CCL4L1, CCL5, CCL7, CCL8, CCR1, CCR2, CCR3, CCR4, CCR7, CD14, CD40, CD40LG, CD74, CD97, CEBPB, CHST1, CIAS1, CKLF, CRP, CX3CL1, CXCL1, CXCL10, CXCL11, CXCL12, CXCL13, CXCL14, CXCL16, CXCL2, CXCL3, CXCL5, CXCL6, CXCL9, CYBB, DOCK2, EPHX2, F11R, FOS, FPR1, GPR68, HDAC4, HDAC5, HDAC7A, HDAC9, HRH1, ICEBERG, IFNA2, IL10, IL10RB, IL13, IL17, IL17B, IL17C, IL17D, IL17E, IL17F, IL18RAP, IL1A, IL1B, 7L1F10, IL1F5, IL1F6, IL1R1, IL1RAP, IL1RN, IL20, IL22, IL31RA, IL5, IL8, IL8RA, IL8RB, IL9, IRAK2, IRF7, ITCH, ITGAL, ITGB2, KNG1, LTA4H, LTB4R, LY64, LY75, LY36, LY96, MEFV, MGLL, MIF, MMP25, MYD38, NALP12, NCR3, NFAM1, NFATC3, NFATC4, NFE2L1, NFKB1, NFRKB, NFX1, NMI, NOS2A, NR3G1, OLR1, PAP, PARP4, PLA2G2D, PLA2G7, PRDX5, PREX1, PRG2, PRG3, PROCR, PROK2, PTAFR, PTGS2, PTPRA, PTX3, REG-III, RIPK2, S100A12, S100A8, SAA2, SCUBE1, SCYE1, SELE, SERPINA3, SFTPD, SN, SPACA3, SPP1, STAB1, SYK, TACR1, TIRAP, TLR1, TLR10, TLR2, TLR3, TLR4, TLR5, TLR6, TLR7, TLR8, TLR9, TNF, TNFAIP6, TOLLIP, TPST1, VPS45A, XCR1.

Humoral Immune Response: BATF, BCL2, BF, BLNK, C1R, C2, C3, C4A, CCL16, CCL18, CCL2, CCL20, CCL22, CCL3, CCL7, CCR2, CCR6, CCR7, CCRL2, CCRL2, CD1B, CD1C, CD22, CD28, CD40, CD53, CD58, CD74, CD86, CLC, CR1, CRLF1, CSF1R, CSF2RB, CXCR3, CYBB, EBI3, FADD, GPI, IL10, IL12A, IL12B, IL12RB1, IL13, IL18, IL1B, IL2, IL26, IL4, IL6, IL7, IL7R, IRF4, ITGB2, LTF, LY86, LY9, LY96, MAPK11, MAPK14, MCP, NFKB1, NR4A2, PAX5, POU2AF1, POU2F2, PTAFR, RFXANK, S100B, SERPING1, SFTPD, SLA2, TNFRSF7, XCL1, XCR1, YY1.

Growth factor and associated molecule: BMP1, BMP2, BMP3, BMP4, BMP5, BMP6, BMP7, BMP8, BMPR1A, CASR, CSF2 (GM-CSF), CSF3 (G-CSF), EGF, EGFR, FGF1, FGF2, FGF3, FGFR1, FGFR2, FGFR3, FLT1, GDF10, IGF1, IGF1R, IGF2, MADH1, MADH2, MADH3, MADH4, MADH5, MADH6, MADH7, MADH9, MSX1, MSX2, NFKB1, PDGFA, RUNX2 (CBFA1), SOX9, TGFB1, TGFB2, TGFB3, TGFBR1, TGFBR2, TNF (TNFa), TWIST, VDR, VEGF, VEGFB, VEGFC

Matrix and its associated protein: ALPL, ANXA5, ARSE, BGLAP (osteocalcin), BGN, CD36, CD36L1, CD36L2, COL1A1, COL2A1, COL3A1, COL4A3, COL4A4, COL4A5, COL5A1, COL7A1, COL9A2, COL10A1, COL11A1, COL12A1, COL14A1, COL15A1, COL16A1, CCL17A1, COL18A1, COL19A1, CTSK, DCN, FN1, MMP2, MMP8, MMP9, MMP10, MMP13, SERPINH1 (CBP1), SERPINH2 (CBP2), SPARC, SPP1 (osteopontin); or

Cell adhesion molecule: ICAM1, ITGA1, ITGA2, ITGA3, ITGAM, ITGAV, ITGB1, VCAM1

The illustrated embodiments of the invention also include a method for in vivo intracellular transfection of gene, siRNA, shRNA vectors, and other biomedical diagnostic and therapeutic drugs and molecules for the treatment of arthritis and other orthopedic diseases in large animals and humans utilizing any one of the apparatus and materials disclosed above.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1a is a lateral side cross section of a spine into which a mesh system is inserted into the vertebral canal by means of a catheter and the LSEN field applied with release of biomaterial.

FIG. 1b is a horizontal cross sectional view of the mesh system of the invention as seen through section lines 1b-1b of FIG. 1a.

FIG. 2 is a cutaway perspective view of the embodiment where the mesh system of the invention is placed beneath and above the flat bones of the skull.

FIG. 3a is front plan view of the sternum where the mesh system of the invention is placed above the breast bone.

FIG. 3b is diagrammatic longitudinal side cross sectional of the sternum application of FIG. 3a.

FIG. 4a is an idealized plan view of the use of LSEN fields in a bone or joint with artificial material. In the first step in the example of a hip joint replacement as depicted in the leftmost view, a tunnel is first made in the femur and the bone treated with a biomaterial and LSEN fields from a mesh system implanted in the tunnel. Thereafter as shown in the rightmost view, the artificial joint is implanted.

FIG. 4b is an idealized plan view the use of LSEN fields in a bone or joint with artificial material. In the first step in the example of a hip joint replacement as depicted in the leftmost view, a tunnel is made in the femur, the artificial joint, a biomaterial and a mesh system implanted in the femur and hip socket. Thereafter as shown in the rightmost view, a mesh system is placed on the outside of the body surface adjacent to the joint location and the tissue treated with LSEN fields with the biomaterial.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The illustrated embodiment of the invention includes: 1) an apparatus for highly efficient in vivo low strength electric field network-mediated localized intracellular transfection of gene, siRNA, shRNA vectors, and other biomedical diagnostic and therapeutic drugs and molecules for the treatment of arthritis and other orthopedic diseases; 2) a methodology for using low strength electric field network-mediated two or more gene, siRNA, shRNA vector, and other biomedical diagnostic and therapeutic drugs and molecules combined therapy in arthritis and other orthopedic diseases; and 3) an exemplary list of the molecules which may be used in this methodology with the disclosed apparatus.

Efficient and safe drug delivery is the key element in the disclosed treatment. It has been known that localized drug delivery not only can result in a significant increase in the concentration of a drug in the targeted tissue and organ and improve the therapeutic efficacy, but also can significantly reduce or avoid the systemic adverse effect of the drug. Because the local concentration of the drug in the targeted tissue or organ is greatly increased, the dose of the drug which is given can be materially decreased, thereby further reducing any possible side effects, whether such effects are whole body and even localized to the treatment site.

Recently, I developed a novel low strength electric field network (LSEN)-mediated drug and gene delivery method for used in tissue and organs of large animal or human. We also designed the apparatus for the joint and bone application. See U.S. Pat. No. 6,593,130, U.S. patent application Ser. No. 11/909,074 corresponding to PCT/US2006/011355, U.S. Patent Application 2005/0119518, U.S. Provisional Patent Applications 60/894,877, and 60/894,831, each incorporated herein by reference.

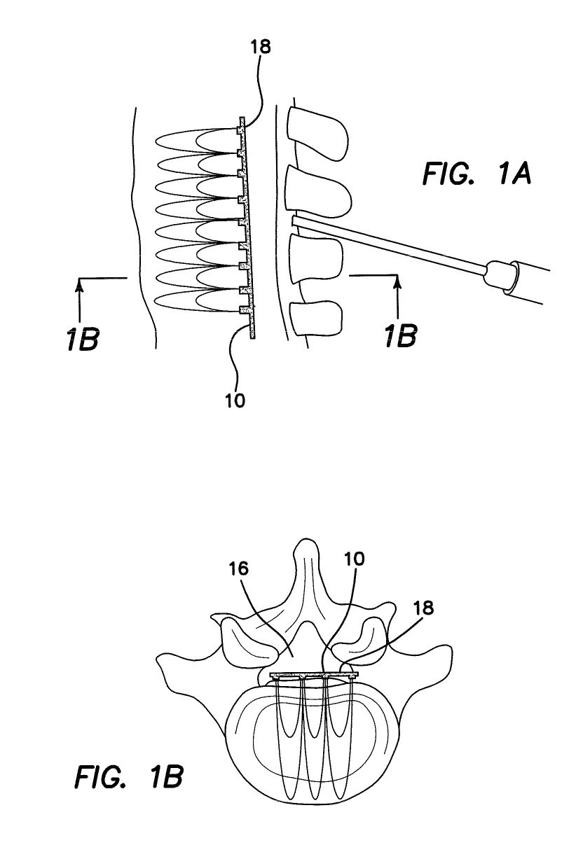

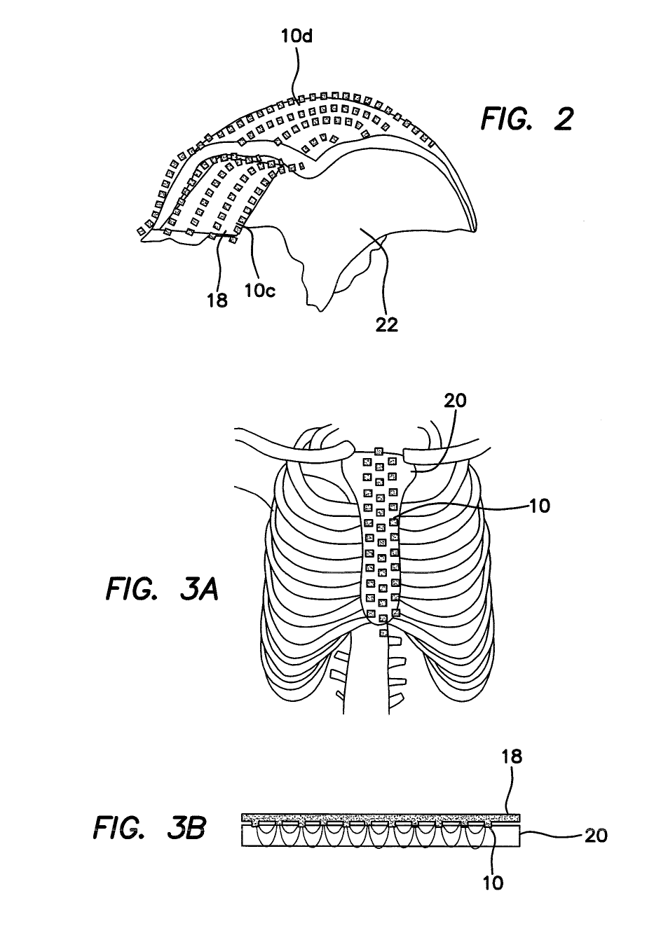

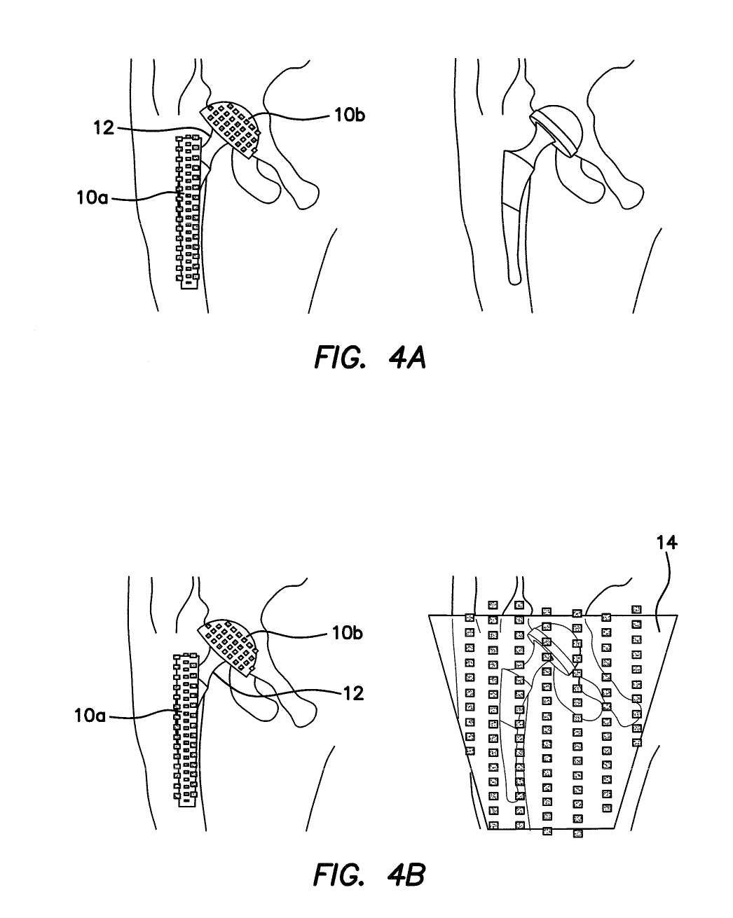

This includes LSEN apparatus for the joint and bone drug delivery, specifically for the spinal drug delivery as depicted in FIGS. 1a and 1b. I also have been able to show the efficient in vivo delivery of siRNA and shRNA delivery in joint as in FIGS. 4a and 4b or in the case of flat bone as shown in FIGS. 2, 3a and 3b. The illustrated embodiment of the invention introduces a new strategy for in vivo intracellular transfection of gene, siRNA, shRNA vectors, and other biomedical diagnostic and therapeutic drugs and molecules for the treatment of arthritis and other orthopedic diseases in large animals or humans. To be able to apply low voltage, short pulse, and long duration bursts (LSEN pulses) into a joint, that includes bones, cartilages, and related tissues, to create a more uniformly distributed electric field network, disclosed below are several drug delivery systems. The details of the LSEN pulses and the structure of the mesh electrode systems which are used are set forth in the incorporated applications and patents and will not be further discussed here except where relevant. What are of primary emphasis are the new applications to which such electrode meshes and LSEN methodologies may be employed. It is to be expressly understood that many different embodiments and equivalent arrangements of the electrode meshes and the LSEN voltages could be employed without departing from the spirit and scope of the disclosed invention.

The applications include LSEN-mediated gene, siRNA, shRNA vector, and other biomedical diagnostic and therapeutic drugs and molecules for the treatment of arthritis and other orthopedic diseases in large animal and/or human joints.

The disclosed LSEN apparatus is comprised of alternatively arranged negative and positive electrodes in an array or arrays 10a, 10b which is inserted into the joint cavity through a catheter or surgically as in the illustration of FIG. 4a. In the illustration mesh 10a is disposed into a tunnel created in the femur and mesh 10b is disposed into the hip socket. LSEN fields may then be applied in the presence of a biomaterial, drug or gene and after treatment the prosthesis 12 implanted in a conventional manner. The illustration shows use during implantation of an artificial hip joint, but the process is similar in the case of a joint which is treated where no prosthesis is implanted.

Alternatively as shown in FIG. 4b, meshes 10a and 10b may be implanted in combination with an either alternatively arranged negative and positive electrodes array or just all negative electrodes array 14 which is positioned outside of the joint and in directly contact with the skin. The LSEN field is then applied using meshes 10a, 10b and 14. Using the system of FIG. 4b, we can generate more uniformly distributed and more dense electric field patterns in the joint which has better gene transfer efficiency. This system may be more suitable for the siRNA and shRNA delivery because gene siRNA and shRNA can be applied into the joint cavity and remain in place for a long period of time. LSEN can be applied for a long time durations using this system as well to give an opportunity for better and more stable transfection for the treatment of arthritis and other joint diseases.

LSEN-mediated gene, siRNA, shRNA vector, and other biomedical diagnostic and therapeutic drugs and molecules for the treatment of arthritis and other orthopedic diseases in large animals or humans' spine as depicted in FIGS. 1a and 1b.

This spinal system includes an alternatively arranged negative and positive electrodes in an array or mesh 10 which is inserted into the vertebral canal 16. A slow drug infusion bag or other agent 18 for releasing materials is fixed to the electrode array 10 and is also used to shield or insulate the electric field from spinal cord. Thus, gene, siRNA, shRNA vectors, and other biomedical diagnostic and therapeutic drugs and molecules can be distributed evenly into the targeted spine. A uniformly distributed electric field network is applied on the targeted spine while gene, siRNA, shRNA vectors, and other biomedical diagnostic and therapeutic drugs are applied for the treatment of spinal diseases.

LSEN-mediated gene, siRNA, shRNA vectors, and other biomedical diagnostic and therapeutic drugs and molecules for the treatment of arthritis and other orthopedic diseases in large animals or human skulls or flat bones as shown in FIGS. 2, 3a and 3b.

This cranial system is comprised of an alternatively arranged negative and positive electrodes in an array or mesh 10c which can be placed on the skull 22. A slow drug infusion bag or other agent 18 for releasing materials is fixed on the electrode array 10c. Alternatively, a negative electrode array or mesh 10d is applied on the outside of the skull 22 and a positive electrode mesh 10c on the cranial side of the skull 22 through a catheter. Again a slow drug infusion bag or other agent 18 for releasing materials is fixed on the cranial side electrode array 10c to shield or insulate the brain from the electric field. Thus, gene, siRNA, shRNA vectors, and other biomedical diagnostic and therapeutic drugs and molecules can be distributed evenly into the targeted skull or bone tissue. A uniformly distributed electric field network can be applied on the targeted bone while gene, siRNA, shRNA vectors, and other biomedical diagnostic and therapeutic drugs are applied for the treatment of skull or other flat bone diseases. FIG. 3a shows a mesh 10 applied to the surface of the sternum with a slow drug infusion bag or other agent 18 for releasing materials disposed outside mesh 10. The longitudinal cross sectional view of FIG. 3b more clearly depicts the placement of the bag 18 relative to sternum 20 and mesh 10.

LSEN-mediated gene, siRNA, shRNA vectors, and other biomedical diagnostic and therapeutic drugs and molecules for the treatment of arthritis and other orthopedic diseases in large animals and human long bones or joints with screws, needles, prosthesis or other artificial material.

The disclosed system is intended for the treatment in long bones or joints while placing the screw, needle, prosthesis or other artificial material. The disclosed LSEN apparatus is comprised of alternatively arranged negative and positive electrodes in an array or arrays 10a, 10b which is inserted into the joint cavity through a catheter or surgically as in the illustration of FIG. 4a in connection with a hip joint prosthesis 12. In the illustration mesh 10a is disposed into a tunnel created in the femur to receive one portion of the prosthesis 12 and mesh 10b is disposed into the hip socket. LSEN fields may then be applied in the presence of a biomaterial, drug or gene and after treatment the prosthesis 12 implanted in a conventional manner.

Alternatively as shown in FIG. 4b, meshes 10a and 10b may be implanted with prosthesis 12 in combination with an either alternatively arranged negative and positive electrodes array or just all negative electrodes array 14 which is positioned outside of the joint and in directly contact with the skin. The LSEN field is then applied using meshes 10a, 10b and 14. Using the system of FIG. 4b, we can generate more uniformly distributed and more dense electric field patterns in the joint which has better gene transfer efficiency. This system may be more suitable for the siRNA and shRNA delivery because gene siRNA and shRNA can be applied into the joint cavity and remain in place for a long period of time. LSEN can be applied for a long time durations using this system as well to give an opportunity for better and more stable transfection for the treatment of arthritis and other joint diseases.

This orthopedic system is comprised of an alternatively arranged negative and positive electrodes in one or more arrays or meshes 10 which are placed in the bones and joints in the position where the screw, needle, prosthesis or other artificial material will be inserted. A slow drug infusion bag or other agent 18 for releasing materials is fixed on the electrode array. A negative electrode array or mesh 14 is positioned on the outside of the joint or long bone Thus, gene, siRNA, shRNA vectors, and other biomedical diagnostic and therapeutic drugs and molecules can be delivered evenly in the targeted joint and bone. A uniformly distributed electric network field (LSEN) can be applied on the targeted joint and long bone while gene, siRNA, shRNA vector, and other biomedical diagnostic and therapeutic drugs are applied for the treatment of bone diseases before the screw, needle, prosthesis or other artificial material is placed.

Set out below is an exemplary list of known molecules and their inhibitors, enhancers, regulators, genes, siRNAs, shRNA s, antigens, antibodies, or peptides related with these molecules, which can be used in the disclosed embodiments for the arthritis and other orthopedic diseases. It must be understood that this listing is not exhaustive and the invention is contemplated as including other molecules now known and later devised which may be electroporated into tissue using the disclosed embodiments.

Cytokines:

a Chemokines: CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCL3L1, CCL4, CCL4L1, CCL5, CCL7, CCL8, CKLF, CX3CL1, CXCL1, CXCL10, CXCL11, CXCL12, CXCL13, CXCL14, CXCL2, CXCL3, CXCL5, CXCL6, CXCL9, CYP26B1, IL13, IL8, PF4V1, PPBP, PXMP2, XCL1.

b. Other Cytokines: AREG, BMP1, BMP2, BMP3, BMP7, CAST, CD40LG, CER1, CKLFSF1, CKLFSF2, CLC, CSF1, CSF2, CSF3, CTF1, CXCL16, EBI3, ECGF1, EDA, EPO, ERBB2, ERBB2IP, FAM3B, FASLG, FGF10, FGF12, FIGF, FLT3LG, GDF2, GDF3, GDF5, GDF6, GDF8, GDF9, GLMN, GPI, GREM1, GREM2, GRN, IFNA1, IFNA14, IFNA2, IFNA4, IFNA8, IFNB1, IFNE1, IFNG, IFNK, IFNW1, IFNWP2, IK, IL10, IL11, IL12A, IL12B, IL15, IL16, IL17, IL17B, IL17C, IL17D, IL17E, IL17F, IL18, IL19, IL1A, IL1B, IL1F10, IL1F5, IL1F6, IL1F7, IL1F8, IL1F9, IL1RN, IL2, IL20, IL21, IL22, IL23A, IL24, IL26, IL27, IL23B, IL29, IL3, IL32, IL4, IL5, IL6, IL7, IL9, INHA, INHBA, INHBB, KITLG, LASS1, LEFTY1, LEFTY2, LIF, LTA, LTB, MDK, MIF, MUC4, NODAL, OSM, PBEF1, PDGFA, PDGFB, PRL, PTN, SCGB1A1, SCGB3A1, SCYE1, SDCBP, SECTM1, SIVA, SLCO1A2, SLURP1, SOCS2, SPP1, SPRED1, SRGAP1, THPO, TNF, TNFRSF11B, TNFSF10, TNFSF11, TNFSF13, TNFSF13B, TNFSF14, TNFSF15, TNFSF18, TNFSF4, TNFSF7, TNFSF8, TNFSF9, TRAP1, VEGF, VEGFB, YARS.

Cytokine Receptors:

a. Cytokine Receptors: CNTFR, CSF2RA, CSF2RB, CSF3R, EBI3, EPOR, F3, GFRA1, GFRA2, GHR, IFNAR1, IFNAR2, IFNGR1, IFNGR2, IL10RA, IL10RB, IL11RA, IL12B, IL12RB1, IL12RB2, IL13RA1, IL13RA2, IL15RA, IL17R, IL17RB, IL18R1, IL1R1, IL1R2, IL1RAP, IL1RAPL2, IL1RL1, IL1RL2, IL20RA, IL21R, IL22RA1, IL22RA2, IL28RA, IL2RA, IL2RB, IL2RG, IL31RA, IL3RA, IL4R, IL5RA, IL6R, IL6ST, IL7R, IL8RA, IL8RB, IL9R, LEPR, LIFR, MPL, OSMR, PRLR, TTN.

b. Chemokine Receptors: BLR1, CCL13, CCR1, CCR10, CCR2, CCR3, CCR4, CCR5, CCR6, CCR7, CCR8, CCR9, CCRL1, CCRL2, CX3CR1, CXCR3, CXCR4, CXCR6, IL8RA, IL8RB, XCR1.

Cytokine Metabolism: APOA2, ASB1, AZU1, B7H3, CD28, CD4, CD80, CD86, EBI3, GLMN, IL10, IL12B, IL17F, IL18, IL21, IL27, IL4, INHA, INHBA, INHBB, IRF4, NALP12, PRG3, S100B, SFTPD, SIGIRR, SPN, TLR1, TLR3, TLR4, TLR6, TNFRSF7, TNFSF15.

Cytokine Production: APOA2, ASB1, AZU1, B7H3, CD28, CD4, CD80, CD36, EBI3, GLMN, IL10, IL12B, IL17F, IL18, IL21, IL27, IL4, INHA, INHBA, INHBB, INS, IRF4, NALP12, NFAM1, NOX5, PRG3, S100B, SAA2, SFTPD, SIGIRR, SPN, TLR1, TLR3, TLR4, TLR6, TNFRSF7.

Other Genes involved in Cytokine-Cytokine Receptor Interaction: ACVR1, ACVR1B, ACVR2, ACVR2B, AMH, AMHR2, BMPR1A, BMPR1B, BMPR2, CCR1, CD40, CRLF2, CSF1R, CXCR3, IL18RAP, IL23R, LEP, TGFB1, TGFB2, TGFB3, TGFBR1, TGFBR2, TNFRSF1A, TNFRSF1B, TNFRSF21, TNFRSF8, TNFRSF9, XCR1.

Acute-Phase Response: AHSG, APGS, APOL2, CEBPB, CRP, F2, F8, FN1, IL22, IL6, INS, ITIH4, LBP, PAP, REG-III, SAA2, SAA3P, SAA4, SERPINA1, SERPINA3, SERPINF2, SIGIRR, STAT3.

Inflammatory Response: ADORA1, AHSG, AIF1, ALOX5, ANXA1, APOA2, APOL3, ATRN, AZU1, BCL6, BDKRB1, BLNK, C3, C3AR1, C4A, CCL1, CCL11, CCL13, CCL16, CCL17, CCL18, CCL19, CCL2, CCL20, CCL21, CCL22, CCL23, CCL24, CCL25, CCL26, CCL3, CCL3L1, CCL4, CCL4L1, CCL5, CCL7, CCL8, CCR1, CCR2, CCR3, CCR4, CCR7, CD14, CD40, OD40LG, CD74, CD97, CEBPB, CHST1, CIAS1, CKLF, CRP, CX3CL1, CXCL1, CXCL10, CXCL11, CXCL12, CXCL13, CXCL14, CXCL16, CXCL2, CXCL3, CXCL5, CXCL6, CXCL9, CYBB, DOCK2, EPHX2, F11R, FOS, FPR1, GPR68, HDAC4, HDAC5, HDAC7A, HDAC9, HRH1, ICEBERG, IFNA2, IL10, IL10RB, IL13, IL17, IL17B, IL17C, IL17D, IL17E, IL17F, IL18RAP, IL1A, IL1B, IL1F10, IL1F5, IL1F6, IL1R1, IL1RAP, IL1RN, IL20, IL22, IL31RA, IL5, IL8, IL8RA, IL8RB, IL9, IRAK2, IRF7, ITCH, ITGAL, ITGB2, KNG1, LTA4H, LTB4R, LY64, LY75, LY86, LY96, MEFV, MGLL, MIF, MMP25, MYD88, NALP12, NCR3, NFAM1, NFATC3, NFATC4, NFE2L1, NFKB1, NFRKB, NFX1, NMI, NOS2A, NR3Cl, OLR1, PAP, PARP4, PLA2G2D, PLA2G7, PRDX5, PREX1, PRG2, PRG3, PROCR, PROK2, PTAFR, PTGS2, PTPRA, PTX3, REG-III, RIPK2, S100A12, S100A8, SAA2, SCUBE1, SCYE1, SELE, SERPINA3, SFTPD, SN, SPACA3, SPP1, STAB1, SYK, TACR1, TIRAP, TLR1, TLR10, TLR2, TLR3, TLR4, TLR5, TLR6, TLR7, TLR8, TLR9, TNF, TNFAIP6, TOLLIP, TPST1, VPS45A, XCR1.

Humoral Immune Response: BATF, BCL2, BF, SLNK, C1R, C2, C3, C4A, CCL16, CCL18, CCL2, CCL20, CCL22, CCL3, CCL7, CCR2, CCR6, CCR7, CCRL2, CCRL2, CD1B, CD1C, CD22, CD28, CD40, CD53, CD58, CD74, CD86, CLC, CR1, CRLF1, CSF1R, CSF2RB, CXCR3, CYBB, EBI3, FADD, GPI, IL10, IL12A, IL12B, IL12RB1, IL13, IL18, IL1B, IL2, IL26, IL4, IL6, IL7, IL7R, IRF4, ITGB2, LTF, LY86, LY9, LY96, MAPK11, MAPK14, MCP, NFKB1, NR4A2, PAX5, POU2AF1, POU2F2, PTAFR, RFXANK, S100B, SERPING1, SFTPD, SLA2, TNFRSF7, XCL1, XCR1, YY1.

Growth factor and associated molecule: BMP1, BM P2, BMP3, BMP4, BMP5, BMP6, BMP7, BMP8, BMPR1A, CASR, CSF2 (GM-CSF), CSF3 (G-CSF), EGF, EOFR, FGF1, FGF2, FGF3, FGFR1, FGFR2, FGFR3, FLT1, GDF10, IGF1, IGF1R, IGF2, MADH1, MADH2, MADH3, MADH4, MADH5, MADH6, MADH7, MADH9, MSX1, MSX2, NFKS1, PDGFA, RUNX2 (CBFA1), SOX9, TFB1, TGFB2, TGFB3, TGFBR1, TGFBR2, TNF (TNFa), TWIST, VDR, VEGF, VEGFB, VEGFC

Matrix and its associated protein: ALPL, ANXA5, ARSE, BGLAP (osteocalcin), BGN, C36, CD36L1, CD36L2, COL1A1, COL2A1, COL3A1, COL4A3, COL4A4, COL4A5, COL5A1, COL7A1, COL9A2, COL10A1, COL11A1, COL12A1, COL14A1, COL15A1, COL16A1, COL17A1, COL18A1, COL19A1, CTSK, DCN, FN1, MMP2, MMP8, MMP9, MMP10, MMP13, SERPINH1 (CBP1), SERPINH2 (CBP2), SPARC, SPP1 (osteopontin)

Cell adhesion molecule: ICAM1, ITGA1, ITGA2, ITGA3, ITGAM, ITGAV, ITGB1, VCAM1

Skeletal Development:

a. Bone Mineralization: AHSG, AMBN, AMELY, BGLAP, ENAM, MGP, MINPP1, SPP1, STATH, TUFT1.

b. Cartilage Condensation: BMP1, COL11A1, MGP, SOX9.

c. Ossification: ALPL, AMBN, AMELY, BGLAP, CALCR, CASR, CDH11, DMP1, DSPP, ENAM, IBSP, MGP, MINPP1, PHEX, RUNX2, SOST, SPARC, SPP1, STATH, TFIP11, TUFT1.

d. Osteoclast Differentiation: BOLAP, TWIST2.

e. Other Genes Involved in Skeletal Development: ARSE, BMP2, BMP3, BMP4, BMP5, BMP6, BMP7, BMP8B, COL10A1, COL12A1, COL1A1, COL1A2, COL2A1, COL9A2, COMP, FGFR1, FGFR3, GDF10, IGF1, IGF2, MSX1, MSX2, TWIST1.

Bone Mineral Metabolism:

a. Calcium on Binding and Homeostasis: ANXA5, ARSE, BGLAP, BMP1, CALCR, CASR, CDH11, COMP, DMP1, EGF, MGP, MMP13, MMP2, MMP8, SPARC, VDR.

b. Phosphate Transport: COL10A1, COL11A1, COL12A1, COL14A1, COL15A1, COL16A1, COL17A1, COL18A1, COL19A1, COL1A1, COL1A2, COL2A1, COL3A1, COL4A3, COL4A4, COL4A5, COL5A1, COL7A1, COL9A2.

Cell Growth and Differentiation:

a. Regulation of the Cell Cycle: EGFR, FGF1, FGF2, FGF3, IGF1R, IGF2, PDGFA, TGFB1, TGFB2, TGFB3, VEGF, VEGFB, VEGFC.

b. Cell Proliferation: COL18A1, COL4A3, CSF3, EGF, EGFR, FGF1, FGF2, FGF3, FLT1, IGF1, IGF1R, IGF2, PDGFA, SMAD3, SPP1, TGFB1, TGFB2, TGFB3, TGFBR2, VEGF, VEGFB, VEGFC.

c. Growth Factors and Receptors BMP1, BMP2, BMP3, BMP4, BMP5, BMP6, BMP7, BMP8B, BMPRP1A, CSF2, CSF3, EGF, EGFR, FGF1, FGF2, FGF3, FGFR1, FGFP2, FGFR3, FLT1, GDF10, IGF1, IGF1R, IGF2, PDGFA, SPP1, TGFB1, TGFB2, TGFB3, TGFBR1, TGFBPR2, VEGF, VEGFB, VEGFC.

d. Cell Differentiation: SPP1, TFIP11, TWIST1, TWIST2.

Extracellular Matrix (ECM) Molecules:

e. Basement Membrane Constituents: COL4A3, COL4A4, COL4A5, COL7A1, SPARC.

f. Collagens: COL10A1, COL11A1, COL12A1, COL14A1 COL15A1, COL16A1, COL18A1, COL19A1, COL1A1, COL1A2, COL2A1, COL3A1, COL4A3, COL4A4, COL4A5, COL5A1, COL7A1, COL9A2.

g. ECM Protease Inhibitors: AHSG, COL4A3, COL7A1, SERPINH1.

ECM Proteases: BMP1, CTSK7 MMP10, MMP13, MMP2, MMP8, MMP9, PHEX

h. Structural Constituents of Bone: BGLAP, COL1A1, COL1A2, MGP.

Structural Constituents of Tooth Enamel: AMBN, AMELY, ENAM, STATH, TUFT1.

Other ECM Molecules: BGN, BMP2, BMP8B, COL17A1, COMP, CSF2, CSF3, DCN, DSPP, EGF, FGF1, FGF2, FGF3, FLT1, GDF10, IBSP, IGF1, IGF2, PDGFA, SPP1, VEGF, VEGFB.

Cell Adhesion Molecules:

a. Cell-cell Adhesion: CDH11, COL11A1, COL14A1, COL19A1, ICAM1, ITGB1, VCAM1.

b. Cell-matrix Adhesion: ITGA1, ITGA2, ITGA3, ITGAM, ITGAV, ITGB1, SPP1.

c. Other Cell Adhesion Molecules: BGLAP, CD36, COL12A1, COL15A1, COL16A1, COL18A1, COL4A3, COL5A1, COL7A1, COMP, FN1, IBSP, SCARB1, TNF.

Transcription Factors and Regulators: MSX1, MSX2, NFKB1, RUNX2, SMAD1, SMAD2, SMAD3, SMAD4, SMAD5, SMAD6, SMAD7, SMAD9, SOX9, TNF, TWIST1, TWIST2, VDR.

This invention opens a new era for the mediated gene, siRNA, shRNA vector, and other biomedical diagnostic and therapeutic drugs and molecules for the treatment of arthritis and other orthopedic diseases in large animals and human. Our recent data have shown the applicability of this technique. There is no existing technique which is applicable for efficient in vivo intracellular gene, siRNA, shRNA vector transfer for human use.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

1. An apparatus for in vivo intracellular transfection of gene, siRNA, shRNA vectors, and other biomedical diagnostic and therapeutic drugs and molecules for the treatment of arthritis and other orthopedic diseases in large animals and humans comprising:
a source of low voltage, short duration pulses in long duration bursts (LSEN);
an electrode mesh system coupled to the source for generating distributed electric field network into a joint, including bones, cartilages, and related tissues; and
means for transfecting the gene, siRNA, shRNA vectors, and other biomedical diagnostic and therapeutic drugs and molecules into a joint
where the electrode mesh system comprises alternatively arranged negative and positive electrodes in a first array which is capable of being inserted into a joint cavity, and either an alternatively arranged negative and positive or an all negative second electrode array which is positioned outside of the joint and in directly contact with overlying skin.

2. The apparatus of claim 1 where the electrode mesh system is capable of being deployed for a chronic treatment period.

3. The apparatus of claim 1 further comprising an all negative second electrode array positioned on the outside of the joint, and
where the means for transfecting comprises a slow drug infusion bag or other agent for releasing materials coupled to the first electrode array.

4. An apparatus for in vivo intracellular transfection of gene, siRNA, shRNA vectors, and other biomedical diagnostic and therapeutic drugs and molecules for the treatment of arthritis and other orthopedic diseases in large animals and humans comprising:
a source of low voltage, short duration pulses in long duration bursts (LSEN);
an electrode mesh system coupled to the source for generating distributed electric field network into a spine; and
means for transfecting the gene, siRNA, shRNA vectors, and other biomedical diagnostic and therapeutic drugs and molecules into the spine, where the electrode mesh system comprises alternatively arranged negative and positive electrodes in a first array which is inserted into a vertebral canal associated with the spine and
where the means for transfecting comprises a slow drug infusion bag or other agent for releasing materials coupled to the electrode array and also used to shield or insulate the spine from the electric field network.

5. An apparatus for in vivo intracellular transfection of gene, siRNA, shRNA vectors, and other biomedical diagnostic and therapeutic drugs and molecules for the treatment of arthritis and other orthopedic diseases in large animals and humans comprising:
a source of low voltage, short duration pulses in long duration bursts (LSEN);
an electrode mesh system coupled to the source for generating distributed electric field network into a skull or flat bone; and
means for transfecting the gene, siRNA, shRNA vectors, and other biomedical diagnostic and therapeutic drugs and molecules into the skull or flat bone, where the electrode mesh system comprises either an alternatively arranged negative and positive electrodes in a first array capable of being placed on the skull or flat bone, or an all negative electrode second array is applied on the outside of the skull or flat bone and an all positive first electrode array on the cranial side of the skull or internal side of the flat bone, and
where the means for transfecting comprises a slow drug infusion bag or other agent for releasing materials coupled to the first electrode array and also to shield or insulate the brain from the electric field network in the case of use on the skull.

6. An apparatus for in vivo intracellular transfection of gene, siRNA, shRNA vectors, and other biomedical diagnostic and therapeutic drugs and molecules for the treatment of arthritis and other orthopedic diseases in large animals and humans comprising:
a source of low voltage, short duration pulses in long duration bursts (LSEN);
an electrode mesh system coupled to the source for generating distributed electric field network into long bones or joints with screws, needles, prosthesis or other artificial material; and
means for transfecting the gene, siRNA, shRNA vectors, and other biomedical diagnostic and therapeutic drugs and molecules into the long bones or joints with screws, needles, prosthesis or other artificial material, where the electrode mesh system is comprised of a alternatively arranged negative and positive electrodes in a first array capable of being placed in or on the bones and joints in the position where the screw, needle, prosthesis or other artificial material will be inserted, and an all negative second electrode array positioned on the outside of the joint or long bone, and where the means for transfecting comprises a slow drug infusion bag or other agent for releasing materials coupled to the first electrode array.

7. The apparatus of claim 6 where means for transfecting includes selected molecules effective for the arthritis and other orthopedic diseases and their inhibitors, enhancers, regulators, genes, siRNAs, shRNAs, antigens, antibodies, or peptides related with these molecules.

8. The apparatus of claim 7 where the selected molecules effective for the arthritis and other orthopedic diseases and their inhibitors, enhancers, regulators, genes, siRNAs, shRNAs, antigens, antibodies, or peptides related with these molecules comprise at least one of:
a. Cytokines:
i. Chemokines: CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCL3L1, CCL4, CCL4L1, CCL5, CCL7, CCL8, CKLF, CX3CL1, CXCL1, CXCL10, CXCL11, CXCL12, CXCL13, CXCL14, CXCL2, CXCL3, CXCL5, CXCL6, CXCL9, CYP26B1, IL13, IL8, PF4V1, PPBP, PXMP2, XCL1.
ii. Other Cytokines: AREG, BMP1, BMP2, BMP3, BMP7, CAST, CD40LG, CER1, CKLFSF1, CKLFSF2, CLC, CSF1, CSF2, CSF3, CTF1, CXCL16, EBI3, ECGF1, EDA, EPO, ERBB2, ERBB2IP, FAM3B, FASLG, FGF10, FGF12, FIGF, FLT3LG, GDF2, GDF3, GDF5, GDF6, GDF8, GDF9, GLMN, GPI, GREM1, GREM2, GRN, IFNA1, IFNA14, IFNA2, IFNA4, IFNA8, IFNB1, IFNE1, IFNG, IFNK, IFNW1, IFNWP2, IK, IL10, IL11, IL12A, IL12B, IL15, IL16, IL17, IL17B, IL17C, IL17D, IL17E, IL17F, IL18, IL19, IL1A, IL1B, IL1F10, IL1F5, IL1F6, IL1F7, IL1F8, IL1F9, IL1RN, IL2, IL20, IL21, IL22, IL23A, IL24, IL26, IL27, IL28B, IL29, IL3, IL32, IL4, IL5, IL6, IL7, IL9, INHA, INHBA, INHBB, KITLG, LASS1, LEFTY1, LEFTY2, LIF, LTA, LTB, MDK, MIF, MUC4, NODAL, OSM, PBEF1, PDGFA, PDGFB, PRL, PTN; SCGB1A1, SCGB3A1, SCYE1, SDCBP, SECTM1, SIVA, SLCO1A2, SLURP1, SOCS2, SPP1, SPRED1, SRGAP1, THPO, TNF, TNFRSF11B, TNFSF10, TNFSF11, TNFSF13, TNFSF13B, TNFSF14, TNFSF15, TNFSF18, TNFSF4, TNFSF7, TNFSF8, TNFSF9, TRAP1, VEGF, VEGFB, YARS.
b. Cytokine Receptors:
i. Cytokine Receptors: CNTFR, CSF2RA, CSF2RB, CSF3R, EBI3, EPOR, F3, GFRA1, GFRA2, GHR, IFNAR1, IFNAR2, IFNGR1, IFNGR2, IL10RA, IL10RB, IL11RA, IL12B, IL12RB1, IL12RB2, IL13RA1, IL13RA2, IL15RA, IL17R, IL17RB, IL18R1, IL1R1, IL1R2, IL1RAP, IL1RAPL2, IL1RL1, IL1RL2, IL20RA, IL21R, IL22RA1, IL22RA2, IL28RA, IL2RA, IL2RB, IL2RG, IL31RA, IL3RA, IL4R, IL5RA, IL6R, IL6ST, IL7R, IL8RA, IL8RB, IL9R, LEPR, LIFR, MPL, OSMR, PRLR, TTN.
ii. Chemokine Receptors: BLR1, CCL13, CCR1, CCR10, CCR2, CCR3, CCR4, CCR5, CCR6, CCR7, CCR8, CCR9, CCRL1, CCRL2, CX3CR1, CXCR3, CXCR4, CXCR6, IL8RA, IL8RB, XCR1.
c. Cytokine Metabolism: APOA2, ASB1, AZU1, B7H3, CD28, CD4, CD80, CD86, EBI3, GLMN, IL10, IL12B, IL17F, IL18, IL21, IL27, IL4, INHA, INHBA, INHBB, IRF4, NALP12, PRG3, S100B, SFTPD, SIGIRR, SPN, TLR1, TLR3, TLR4, TLR6, TNFRSF7, TNFSF15.
d. Cytokine Production: APOA2, ASB1, AZU1, B7H3, CD28, CD4, CD80, CD86, EBI3, GLMN, IL10, IL12B, IL17F, IL18, IL21, IL27, IL4, INHA, INHBA, INHBB, INS, IRF4, NALP12, NFAM1, NOX5, PRG3, S100B, SAA2, SFTPD, SIGIRR, SPN, TLR1, TLR3, TLR4, TLR6, TNFRSF7.
e. Other Genes involved in Cytokine-Cytokine Receptor Interaction: ACVR1, ACVR1B, ACVR2, ACVR2B, AMH, AMHR2, BMPR1A, BMPR1B, BMPR2, CCR1, CD40, CRLF2, CSF1R, CXCR3, IL18RAP, IL23R, LEP, TGFB1, TGFB2, TGFB3, TGFBR1, TGFBR2, TNFRSF1A, TNFRSF1B, TNFRSF21, TNFRSF8, TNFRSF9, XCR1.
f. Acute-Phase Response: AHSG, APCS, APOL2, CEBPB, CRP, F2, F8, FN1, IL22, IL6, INS, ITIH4, LBP, PAP, REG-III, SAA2, SAA3P, SAA4, SERPINA1, SERPINA3, SERPINF2, SIGIRR, STAT3.
g. Inflammatory Response: ADORA1, AHSG, AIF1, ALOX5, ANXA1, APOA2, APOL3, ATRN, AZU1, BCL6, BDKRB1, BLNK, C3, C3AR1, C4A, CCL1, CCL11, CCL13, CCL16, CCL17, CCL18, CCL19, CCL2, CCL20, CCL21, CCL22, CCL23, CCL24, CCL25, CCL26, CCL3, CCL3L1, CCL4, CCL4L1, CCL5, CCL7, CCL8, CCR1, CCR2, CCR3, CCR4, CCR7, CD14, CD40, CD40LG, CD74, CD97, CEBPB, CHST1, CIAS1, CKLF, CRP, CX3CL1, CXCL1, CXCL10, CXCL11, CXCL12, CXCL13, CXCL14, CXCL16, CXCL2, CXCL3, CXCL5, CXCL6, CXCL9, CYBB, DOCK2, EPHX2, F11R, FOS, FPR1, GPR68, HDAC4, HDAC5, HDAC7A, HDAC9, HRH1, ICEBERG, IFNA2, IL10, IL10RB, IL13, IL17, IL17B, IL17C, IL17D, IL17E, IL17F, IL18RAP, IL1A, IL1B, IL1F10, IL1F5, IL1F6, IL1R1, IL1RAP, IL1RN, IL20, 1L22, IL31RA, IL5, IL8, IL8RA, IL8RB, IL9, IRAK2, IRF7, ITCH, ITGAL, ITGB2, KNG1, LTA4H, LTB4R, LY64, LY75, LY86, LY96, MEFV, MGLL, MIF, MMP25, MYD88, NALP12, NCR3, NFAM1, NFATC3, NFATC4, NFE2L1, NFKB1, NFRKB, NFX1, NMI, NOS2A, NR3C1, OLR1, PAP, PARP4, PLA2G2D, PLA2G7, PRDX5, PREX1, PRG2, PRG3, PROCR, PROK2, PTAFR, PTGS2, PTPRA, PTX3, REG-III, RIPK2, S100A12, S100A8, SAA2, SCUBE1, SCYE1, SELE, SERPINA3, SFTPD, SN, SPACA3, SPP1, STAB1, SYK, TACR1, TIRAP, TLR1, TLR10, TLR2, TLR3, TLR4, TLR5, TLR6, TLR7, TLR8, TLR9, TNF, TNFAIP6, TOLLIP, TPST1, VPS45A, XCR1.
h. Humoral Immune Response: BATF, BCL2, BF, BLNK, C1R, C2, C3, C4A, CCL16, CCL18, CCL2, CCL20, CCL22, CCL3, CCL7, CCR2, CCR6, CCR7, CCRL2, CCRL2, CD1B, CD1C, CD22, CD28, CD40, CD53, CD58, CD74, CD86, CLC, CR1, CRLF1, CSF1R, CSF2RB, CXCR3, CYBB, EBI3, FADD, GPI, IL10, IL12A, IL12B, IL12RB1, IL13, IL18, IL1B, IL2, IL26, IL4, IL6, IL7, IL7R, IRF4, ITGB2, LTF, LY86, LY9, LY96, MAPK11, MAPK14, MCP, NFKB1, NR4A2, PAX5, POU2AF1, POU2F2, PTAFR, RFXANK, S100B, SERPING1, SFTPD, SLA2, TNFRSF7, XCL1, XCR1, YY1.
i. Growth factor and associated molecule: BMP1, BMP2, BMP3, BMP4, BMP5, BMP6, BMP7, BMP8, BMPR1A, CASR, CSF2 (GM-CSF), CSF3 (G-CSF), EGF, EGFR, FGF1, FGF2, FGF3, FGFR1, FGFR2, FGFR3, FLT1, GDF10, IGF1, IGF1R, IGF2, MADH1, MADH2, MADH3, MADH4, MADH5, MADH6, MADH7, MADH9, MSX1, MSX2, NFKB1, PDGFA, RUNX2 (CBFA1), SOX9, TGFB1, TGFB2, TGFB3, TGFBR1, TGFBR2, TNF (TNFa), TWIST, VDR, VEGF, VEGFB, VEGFC
j. Matrix and its associated protein: ALPL, ANXA5, ARSE, BGLAP (osteocalcin), BGN, CD36, CD36L1, CD36L2, COL1A1, COL2A1, COL3A1, COL4A3, COL4A4, COL4A5, COL5A1, COL7A1, COL9A2, COL10A1, COL11A1, COL12A1, COL14A1, COL15A1, COL16A1, COL17A1, COL18A1, COL19A1, CTSK, DCN, FN1, MMP2, MMP8, MMP9, MMP10, MMP13, SERPINH1 (CBP1), SERPINH2 (CBP2), SPARC, SPP1 (osteopontin); or
k. Cell adhesion molecule: ICAM1, 1TGA1, ITGA2, ITGA3, ITGAM, ITGAV, ITGB1, VCAM1.

9. An method in vivo intracellular transfection of gene, siRNA, shRNA vectors, and other biomedical diagnostic and therapeutic drugs and molecules for the treatment of arthritis and other orthopedic diseases in large animals and humans comprising:
generating low voltage, short duration pulses in long duration bursts (LSEN);
defining a distributed electric network field in a joint, including bones, cartilages, and related tissues through an implanted electrode mesh system; and
transfecting the gene, siRNA, shRNA vectors, and other biomedical diagnostic and therapeutic drugs and molecules into a joint
where the distributed electric network field is defined by an electrode mesh system comprising alternatively arranged negative and positive electrodes in a first array which is capable of being inserted into a joint cavity, and either an alternatively arranged negative and positive or an all negative second electrode array which is positioned outside of the joint and in directly contact with overlying skin.

10. The method of claim 9 further comprising defining the distributed electric network field by use of an all negative second electrode array positioned on the outside of the joint, and
where transfecting comprises using a slow drug infusion bag or other agent for releasing materials coupled to the first electrode array.

11. A method in vivo intracellular transfection of gene, siRNA, shRNA vectors, and other biomedical diagnostic and therapeutic drugs and molecules for the treatment of arthritis and other orthopedic diseases in large animals and humans comprising:
generating low voltage, short duration pulses in long duration bursts (LSEN);
defining a distributed electric network field in a spine through an implanted electrode mesh system; and
transfecting the gene, siRNA, shRNA vectors, and other biomedical diagnostic and therapeutic drugs and molecules into the spine,
where the distributed electric network field is defined by an electrode mesh system comprises alternatively arranged negative and positive electrodes in a first array which is inserted into a vertebral canal associated with the spine and
where transfecting comprises using a slow drug infusion bag or other agent for releasing materials coupled to the electrode array and also used to shield or insulate the spine from the electric field network.

12. A method in vivo intracellular transfection of gene, siRNA, shRNA vectors, and other biomedical diagnostic and therapeutic drugs and molecules for the treatment of arthritis and other orthopedic diseases in large animals and humans comprising:
generating low voltage, short duration pulses in long duration bursts (LSEN);
defining a distributed electric network field in a skull or flat bone through an implanted electrode mesh system; and
transfecting the gene, siRNA, shRNA vectors, and other biomedical diagnostic and therapeutic drugs and molecules into the skull or flat bone,
where the distributed electric network field is defined by an electrode mesh system comprises either an alternatively arranged negative and positive electrodes in a first array capable of being placed on the skull or flat bone, or an all negative electrode second array is applied on the outside of the skull or flat bone and an all positive first electrode array on the cranial side of the skull or internal side of the flat bone, and
where transfecting comprises using a slow drug infusion bag or other agent for releasing materials coupled to the first electrode array and also to shield or insulate the brain from the electric field network in the case of use on the skull.

13. A method in vivo intracellular transfection of gene, siRNA, shRNA vectors, and other biomedical diagnostic and therapeutic drugs and molecules for the treatment of arthritis and other orthopedic diseases in large animals and humans comprising:
generating low voltage, short duration pulses in long duration bursts (LSEN);
defining a distributed electric network field in long bones or joints with screws, needles, prosthesis or other artificial material joint through an implanted electrode mesh system; and
transfecting the gene, siRNA, shRNA vectors, and other biomedical diagnostic and therapeutic drugs and molecules into the long bones or joints with screws, needles, prosthesis or other artificial material,
where the distributed electric network field is defined by an electrode mesh system is comprised of a alternatively arranged negative and positive electrodes in a first array capable of being placed in or on the bones and joints in the position where the screw, needle, prosthesis or other artificial material will be inserted, and an all negative second electrode array positioned on the outside of the joint or long bone, and
where transfecting comprises using a slow drug infusion bag or other agent for releasing materials coupled to the first electrode array.

14. The method of claim 13 where transfecting includes transfecting selected molecules effective for the arthritis and other orthopedic diseases and their inhibitors, enhancers, regulators, genes, siRNAs, shRNA s, antigens, antibodies, or peptides related with these molecules.

15. The method of claim 14 where transfecting the selected molecules effective for the arthritis and other orthopedic diseases and their inhibitors, enhancers, regulators, genes, siRNAs, shRNA s, antigens, antibodies, or peptides related with these molecules comprise transfecting at least one of:
a. Cytokines:
i. Chemokines: CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCL3L1, CCL4, CCL4L1, CCL5, CCL7, CCL8, CKLF, CX3CL1, CXCL1, CXCL10, CXCL11, CXCL12, CXCL13, CXCL14, CXCL2, CXCL3, CXCL5, CXCL6, CXCL9, CYP26B1, IL13, IL8, PF4V1, PPBP, PXMP2, XCL1.
ii. Other Cytokines: AREG, BMP1, BMP2, BMP3, BMP7, CAST, CD40LG, CER1, CKLFSF1, CKLFSF2, CLC, CSF1, CSF2, CSF3, CTF1, CXCL16, EBI3, ECGF1, EDA, EPO, ERBB2, ERBB21P, FAM3B, FASLG, FGF10, FGF12, FIGF, FLT3LG, GDF2, GDF3, GDF5, GDF6, GDF8, GDF9, GLMN, GPI, GREM1, GREM2, GRN, IFNA1, IFNA14, IFNA2, IFNA4, IFNA8, IFNB1, IFNE1, IFNG, IFNK, IFNW1, IFNWP2, IK, IL10, IL11, IL12A, IL12B, IL15, IL16, IL17, IL17B, IL17C, IL17D, IL17E, IL17F, IL18, IL19, IL1A, IL1B, IL1F10, IL1F5, IL1F6, IL1F7, IL1F8, IL1F9, IL1RN, IL2, IL20, IL21, IL22, IL23A, IL24, IL26, IL27, IL28B, IL29, IL3, IL32, IL4, IL5, IL6, IL7, IL9, INHA, INHBA, INHBB, KITLG, LASS1, LEFTY1, LEFTY2, LIF, LTA, LTB, MDK, MIF, MUC4, NODAL, OSM, PBEF1, PDGFA, PDGFB, PRL, PTN, SCGB1A1, SCGB3A1, SCYE1, SDCBP, SECTM1, SIVA, SLCO1A2, SLURP1, SOCS2, SPP1, SPRED1, SRGAP1, THPO, TNF, TNFRSF11B, TNFSF10, TNFSF11, TNFSF13, TNFSF13B, TNFSF14, TNFSF15, TNFSF18, TNFSF4, TNFSF7, TNFSF8, TNFSF9, TRAP1, VEGF, VEGFB, YARS.
b. Cytokine Receptors:
i. Cytokine Receptors: CNTFR, CSF2RA, CSF2RB, CSF3R, EB13, EPOR, F3, GFRA1, GFRA2, GHR, IFNAR1, IFNAR2, IFNGR1, IFNGR2, IL10RA, IL10RB, IL11RA, IL12B, IL12RB1, IL12RB2, IL13RA1, IL13RA2, IL15RA, IL17R, IL17RB, IL18R1, IL1R1, IL1R2, IL1RAP, IL1RAPL2, IL1RL1, IL1RL2, IL20RA, IL21R, IL22RA1, IL22RA2, IL28RA, IL2RA, IL2RB, IL2RG, IL31RA, IL3RA, IL4R, IL5RA, IL6R, IL6ST, IL7R, IL8RA, IL8RB, IL9R, LEPR, LIFR, MPL, OSMR, PRLR, TTN.
ii. Chemokine Receptors: BLR1, CCL13, CCR1, CCR10, CCR2, CCR3, CCR4, CCR5, CCR6, CCR7, CCR8, CCR9, CCRL1, CCRL2, CX3CR1, CXCR3, CXCR4, CXCR6, IL8RA, IL8RB, XCR1.
c. Cytokine Metabolism: APOA2, ASB1, AZU1, B7H3, CD28, CD4, CD80, CD86, EBI3, GLMN, IL10, IL12B, IL17F, IL18, IL21, IL27, IL4, INHA, INHBA, INHBB, IRF4, NALP12, PRG3, S100B, SFTPD, SIGIRR, SPN, TLR1, TLR3, TLR4, TLR6, TNFRSF7, TNFSF15.
d. Cytokine Production: APOA2, ASB1, AZU1, B7H3, CD28, CD4, CD80, CD86, EBI3, GLMN, IL10, IL12B, IL17F, IL18, 1L21, IL27, IL4, INHA, INHBA, INHBB, INS, IRF4, NALP12, NFAM1, NOX5, PRG3, S100B, SM2, SFTPD, SIGIRR, SPN, TLR1, TLR3, TLR4, TLR6, TNFRSF7.
e. Other Genes involved in Cytokine-Cytokine Receptor Interaction: ACVR1, ACVR1B, ACVR2, ACVR2B, AMH, AMHR2, BMPR1A, BMPR1B, BMPR2, CCR1, CD40, CRLF2, CSF1R, CXCR3, IL18RAP, IL23R, LEP, TGFB1, TGFB2, TGFB3, TGFBR1, TGFBR2, TNFRSF1A, TNFRSF1B, TNFRSF21, TNFRSF8, TNFRSF9, XCR1.
f. Acute-Phase Response: AHSG, APCS, APOL2, CEBPB, CRP, F2, F8, FN1, IL22, IL6, INS, ITIH4, LBP, PAP, REG-III, SAA2, SM3P, SAA4, SERPINA1, SERPINA3, SERPINF2, SIGIRR, STAT3.
g. Inflammatory Response: ADORA1, AHSG, AIF1, ALOX5, ANXA1, APOA2, APOL3, ATRN, AZU1, BCL6, BDKRB1, BLNK, C3, C3AR1, C4A, CCL1, CCL11, CCL13, CCL16, CCL17, CCL18, CCL19, CCL2, CCL20, CCL21, CCL22, CCL23, CCL24, CCL25, CCL26, CCL3, CCL3L1, CCL4, CCL4L1, CCL5, CCL7, CCL8, CCR1, CCR2, CCR3, CCR4, CCR7, CD14, CD40, CD40LG, CD74, CD97, CEBPB, CHST1, CIAS1, CKLF, CRP, CX3CL1, CXCL1, CXCL10, CXCL11, CXCL12, CXCL13, CXCL14, CXCL16, CXCL2, CXCL3, CXCL5, CXCL6, CXCL9, CYBB, DOCK2, EPHX2, F11R, FOS, FPR1, GPR68, HDAC4, HDAC5, HDAC7A, HDAC9, HRH1, ICEBERG, IFNA2, IL10, IL10RB, IL13, IL17, IL17B, IL17C, IL17D, IL17E, IL17F, IL18RAP, IL1A, IL1B, IL1F10, IL1F5, IL1F6, IL1R1, IL1RAP, IL1RN, IL20, IL22, IL31RA, IL5, IL8, IL8RA, IL8RB, IL9, IRAK2, IRF7, ITCH, ITGAL, ITGB2, KNG1, LTA4H, LTB4R, LY64, LY75, LY86, LY96, MEFV, MGLL, MIF, MMP25, MYD88, NALP12, NCR3, NFAM1, NFATC3, NFATC4, NFE2L1, NFKB1, NFRKB, NFX1, NMI, NOS2A, NR3C1, OLR1, PAP, PARP4, PLA2G2D, PLA2G7, PRDX5, PREX1, PRG2, PRG3, PROCR, PROK2, PTAFR, PTGS2, PTPRA, PTX3, REG-III, RIPK2, S100A12, S100A8, SAA2, SCUBE1, SCYE1, SELE, SERPINA3, SFTPD, SN, SPACA3, SPP1, STAB1, SYK, TACR1, TIRAP, TLR1, TLR10, TLR2, TLR3, TLR4, TLR5, TLR6, TLR7, TLR8, TLR9, TNF, TNFAIP6, TOLLIP, TPST1, VPS45A, XCR1.
h. Humoral Immune Response: BATF, BCL2, BF, BLNK, C1R, C2, C3, C4A, CCL16, CCL18, CCL2, CCL20, CCL22, CCL3, CCL7, CCR2, CCR6, CCR7, CCRL2, CCRL2, CD1B, CD1C, CD22, CD28, CD40, CD53, CD58, CD74, CD86, CLC, CR1, CRLF1, CSF1R, CSF2RB, CXCR3, CYBB, EBI3, FADD, GPI, IL10, IL12A, IL12B, IL12RB1, IL13, IL18, IL1B, IL2, IL26, IL4, IL6, IL7, IL7R, IRF4, ITGB2, LTF, LY86, LY9, LY96, MAPK11, MAPK14, MCP, NFKB1, NR4A2, PAX5, POU2AF1, POU2F2, PTAFR, RFXANK, S100B, SERPING1, SFTPD, SLA2, TNFRSF7, XCL1, XCR1, YY1.
i. Growth factor and associated molecule: BMP1, BMP2, BMP3, BMP4, BMP5, BMP6, BMP7, BMP8, BMPR1A, CASR, CSF2 (GM-CSF), CSF3 (G-CSF), EGF, EGFR, FGF1, FGF2, FGF3, FGFR1, FGFR2, FGFR3, FLT1, GDF10, IGF1, IGF1R, IGF2, MADH1, MADH2, MADH3, MADH4, MADH5, MADH6, MADH7, MADH9, MSX1, MSX2, NFKB1, PDGFA, RUNX2 (CBFA1), SOX9, TGFB1, TGFB2, TGFB3, TGFBR1, TGFBR2, TNF (TNFa), TWIST, VDR, VEGF, VEGFB, VEGFC
j. Matrix and its associated protein: ALPL, ANXA5, ARSE, BGLAP (osteocalcin), BGN, CD36, CD36L1, CD36L2, COL1A1, COL2A1, COL3A1, COL4A3, COL4A4, COL4A5, COL5A1, COL7A1, COL9A2, COL10A1, COL11A1, COL12A1, COL14A1, COL15A1, COL16A1, COL17A1, COL18A1, COL19A1, CTSK, DCN, FN1, MMP2, MMP8, MMP9, MMP10, MMP13, SERPINH1 (CBP1), SERPINH2 (CBP2), SPARC, SPP1 (osteopontin); or
k. Cell adhesion molecule: ICAM1, ITGA1, ITGA2, ITGA3, ITGAM, ITGAV, ITGB1, VCAM1.
